(19) United States
(12) Patent Application Publication
(10) Pub. No.: US 2026/0047878 A1
(43) Pub. Date: Feb. 19, 2026

(54) VERTEBRAL DENERVATION IN CONJUNCTION WITH VERTEBRAL FUSION

(71) Applicant: Relievant Medsystems, Inc., Edina, MN (US)

(72) Inventors: Brian W. Donovan, San Jose, CA (US); Ray M. Baker, San Clemente, CA (US)

(21) Appl. No.: 19/370,300

(22) Filed: Oct. 27, 2025

Related U.S. Application Data
(63) Continuation of application No. 18/003,760, now Pat. No. 12,458,428.
(60) Provisional application No. 63/050,295, filed on Jul. 10, 2020.

(51) Int. Cl. A61B 18/12; A61B 17/70; A61B 18/00; A61B 18/02; A61B 18/08; A61B 18/14; A61B 18/18; A61B 18/20; A61F 2/44; A61F 2/46; A61N 7/00

(52) U.S. Cl. CPC A61B 18/1206; A61B 17/7002; A61B 17/7032; A61B 17/7059; A61B 17/7061; A61B 17/7074; A61B 18/02; A61B 18/082; A61B 18/148; A61B 18/1815; A61F 2/4455; A61F 2/4611; A61N 7/00; A61B 2018/00434; A61B 2018/00577; A61B 2018/0293; A61B 2018/126; A61B 2018/1869; A61B 18/20; A61B 2018/2005; A61N 2007/003

(57) ABSTRACT

Described herein are various implementations of systems and methods for treating back pain (e.g., chronic low back pain) caused by different (e.g., independent) sources of pain, such as pain originating or stemming from intervertebral discs, from vertebral endplates, and/or from intraosseous locations within one or more vertebral bodies. For examples, methods for treating back pain (e.g., chronic low back pain) may involve both vertebral fusion (e.g., arthrodesis or spondylodesis to fuse adjacent vertebrae) and neuromodulation (for example, ablation of nerves within or surrounding one or more of the adjacent vertebrae). The neuromodulation may facilitate treatment of pain that is generated by insertion of fusion hardware.

### RELATED APPLICATIONS

This application is a continuation of U.S. patent application Ser. No. 18/003,760 filed Dec. 29, 2022, which is a national stage application of PCT Application No. PCT/US2021/040843 filed Jul. 8, 2021, which claims priority to U.S. Provisional Application No. 63/050,295 filed Jul. 10, 2020, which are incorporated by reference herein in their entirety.

### FIELD

Described herein are various implementations of systems and methods for treating back pain (e.g., chronic low back pain) involving both vertebral fusion (e.g., arthrodesis or spondylodesis to fuse adjacent vertebrae) and neuromodulation (for example, ablation of nerves within or surrounding one or more of the adjacent vertebrae) and/or for treating back pain (e.g., chronic low back pain) caused by different (e.g., independent) sources of pain.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs. The back pain may be acute or chronic, Existing treatments for chronic back pain vary widely and include physical therapy and exercise, chiropractic treatments, injections, rest, pharmacological therapy such as opioids, pain relievers or anti-inflammatory medications, and surgical intervention such as discectomy (e.g., total disc replacement), or disc repair. Existing treatments can be costly, addictive (e.g., opioid dependence), temporary, ineffective, and/or can increase the pain or require long recovery times. In addition, existing treatments may not provide adequate relief for the majority of patients and only a small percentage are surgically eligible.

### SUMMARY

Back pain can be caused by multiple different (e.g., independent) sources (e.g., radicular pain, neuropathic pain). A particular type of treatment may treat one of the different (e.g., independent) sources of pain (e.g., discogenic pain or pain caused by nerve root compression) but not the other (e.g., pain stemming from one or more vertebrae, such as pain from sources in or on the vertebral endplates or in intraosseous locations within one or more vertebrae). In addition, treatment of one of the independent sources of pain could exacerbate, aggravate, or result in pain caused by another different source of pain. According to several embodiments, methods of providing complete pain treatment of multiple independent sources of back pain in a single combined therapeutic procedure (e.g., one surgical intervention or patient visit) are disclosed herein. In some embodiments, the combined therapeutic procedure disclosed herein reduces or eliminates the need for, or dependence on, pharmaceuticals, such as long-term use of opioids. In some embodiments, different parts or portions of the combined therapeutic procedure are performed under the direction of, and using devices or systems of, different entities. In other embodiments, a single entity may instruct performance of the entire combined therapeutic procedure.

Spinal stabilization or fusion (e.g., spondylodesis or arthrodesis) is a surgical procedure in which two or more adjacent vertebrae of the spine are joined (e.g., fused) together to form a solid integrated bone segment with little or no space between the adjacent vertebrae. Fusion may be performed with an intent to provide increased stabilization and/or to reduce pain (e.g., neuropathic pain). Although spinal stabilization or fusion is generally intended to reduce pain (e.g., chronic low back pain, neuropathic pain, radicular pain) caused by degenerative intervertebral discs, vertebral fractures, scoliosis, stenosis, or other spinal issues, spinal fusion procedures may actually result in additional pain (e.g., a different source of pain). The additional pain may be caused, for example, by, or result from, the hardware, or instrumentation, used to accomplish the fusion (e.g., plates, screws, rods, cages) and/or the graft material (e.g., autograft, allograft, bone graft substitutes, and/or bone morphogenetic proteins) inserted between or adjacent the vertebrae.

For example, the hardware may change the biomechanics of the bone at the spine level and result in offsetting of the load and/or loss of bending moments to different portions of the spine or vertebrae. Bone remodeling around screws of the hardware may also offset the load in the spine, thereby resulting in increased pain. The pain may include persistent pain at the bone graft site, pain to vertebrae adjacent the fused vertebrae resulting from additional stress or loading on the bones adjacent to the fused vertebrae, and/or pain caused by damage to, compression, or aggravation of, nerves in or surrounding the vertebral body by the hardware. The graft material positioned between, within, or surrounding the vertebral bodies may exacerbate vertebral endplate pain as a result of increased loading and stress on the endplate(s). The fusion procedure may include scraping or bloodying of one or more of the endplates (for example, if allograft is used), thereby causing increased endplate pain. The fused vertebrae or adjacent vertebrae may potentially result in Modic changes (or symptoms associated therewith) caused by the fusion or the fused vertebrae or adjacent vertebrae may have had pre-existing Modic changes (or symptoms associated therewith) prior to the fusion procedure.

In addition, the stabilization or fusion procedure may only treat one source of pain and not a second independent source of pain. In accordance with several embodiments, neuromodulation (e.g., denervation) procedures may be performed in conjunction with, or in combination with, fusion procedures (e.g., concurrent with, prior to, or after fusion procedures) to reduce the likelihood of, lingering pain (e.g., chronic low back pain). In some embodiments, neuromodulation therapy is performed first (prior to introduction of fusion hardware) by accessing the vertebral body using access instruments inserted transpedicularly through one or both pedicles or extrapedicularly and then applying neuromodulation therapy within the vertebral body. Pedicle screws may then be inserted through one or more access channels (e.g., channels in one or more pedicles) formed by the access instruments. In accordance with several embodiments, the combined single therapeutic procedure may provide synergistic effects or results that would not be achieved by performing either fusion or neuromodulation procedures in isolation. For example, the neuromodulation may advantageously address the above-described pain caused by, or generated as a result of, the stabilization or fusion. In addition, the source of pain (e.g., back pain caused by instability or nerve root compression or slipping of discs or bones of the spine) intended to be treated by spinal fusion may be different than, and independent from, the source of pain intended to be treated by neuromodulation (e.g., back pain caused by degradation of endplates or by vertebral defects or degeneration). Accordingly, in accordance with some embodiments, performing either spinal fusion or neuromodulation (e.g., basivertebral nerve ablation) in isolation may not adequately address all sources of pain and may therefore provide incomplete treatment and reduced patient satisfaction. However, performing both fusion and neuromodulation (e.g., in a single combined therapeutic procedure in one visit, or with one surgical intervention) may advantageously address both or all independent sources of chronic low back pain, thereby increasing patient comfort and satisfaction. In some implementations, both fusion and neuromodulation are performed without closing an open surgical access site between the two procedures. In accordance with several implementations, the combined therapies do not require trialing and feedback to determine appropriate stimulation or other neuromodulation parameters.

In several embodiments, the synergy achieved by combined fusion and neuromodulation reduces a patient's pain score (e.g., Oswestry Disability Index (ODI) score or Visual Analogue Score (VAS)) in a manner that is more favorable than what would be seen additively. Synergistic interactions result in enhanced potency and/or efficacy of the procedures in some embodiments. For example, pain may be improved both short term and long term, the time to return to normal activities may be reduced, and/or the length of time that pain is reduced is increased.

If neuromodulation is performed before fusion, the neuromodulation may be performed within a few minutes (e.g., 5 minutes-30 minutes) or within hours (e.g., less than 1 hour, less than 2 hours, less than 3 hours) prior to the fusion. If neuromodulation is performed following fusion, the neuromodulation may be performed within a few minutes, within a few hours, within a few days, or within a few months following the fusion procedure. For example, the neuromodulation may be performed if pain persists following the fusion procedure. However, neuromodulation may advantageously be performed during the same time as the fusion (e.g., during the same surgical intervention or patient visit) to preempt the likely pain caused by, or resulting from, the fusion procedure, thereby advantageously reducing the number of visits to the doctor and the amount of pain experience by a patient. The neuromodulation may include denervation (e.g., ablation) of one or more intraosseous nerves (e.g., basivertebral nerve or BVN) within one or more vertebral bodies (e.g., the fused vertebrae and/or adjacent vertebrae). In some embodiments, vertebral neuromodulation and fusion are performed under the direction of, and using devices or systems of, different entities. In other embodiments, a single entity may instruct performance of the entire combined therapeutic procedure.

Pain can be very subjective based on individual patient pain tolerance and perception. Additionally, it can be difficult to assess or quantify efficacy of pain treatment based on patient feedback, especially immediately following a fusion procedure because the patients are typically under general anesthesia and unable to provide feedback. In other cases, the patient may be awake but unable to accurately assess pain.

Neuromodulation procedures may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not limited to nerves and may include effects on other tissue, such as tumors or other soft tissue.

In several embodiments, the systems and methods described herein are provided without any electrical stimulation (e.g., without dorsal root ganglion stimulation or without stimulation to promote fusion). In some embodiments, the systems and methods described herein do not involve modulation of facet joints or sacroiliac joints or nerves associated with facet joints or sacroiliac joints.

In accordance with several embodiments, a method of treating multiple independent sources of low back pain of a patient during a single therapeutic intervention includes performing a spinal fusion procedure between two adjacent vertebrae of the patient, wherein the spinal fusion procedure comprises leaving one or more hardware components within or surrounding each of the adjacent vertebrae. The method also includes inserting an access tool within an inner cancellous bone region of a vertebral body of one of the two adjacent vertebrae while avoiding contact with the one or more hardware components. The access tool includes a lumen extending therethrough to facilitate access to the inner cancellous bone region. The method further includes inserting a neuromodulation device through the lumen of the access tool until at least a distal end of the neuromodulation device extends beyond the access tool within the inner cancellous bone region. The method also includes modulating a basivertebral nerve within the inner cancellous bone region using the neuromodulation device and removing the neuromodulation device and the access tool from the patient.

The step of inserting the access tool within the inner cancellous bone region may include extrapedicularly inserting the access tool within the inner cancellous bone region using an access location superior or inferior to a pedicle of a superior vertebra of the adjacent vertebrae. The method may include identifying the two adjacent vertebrae by determining (e.g., via imaging or via biomarkers) that the two adjacent vertebrae exhibit one or more Modic changes or pre-Modic changes, or symptoms indicative of vertebral degeneration or endplate defects. The two adjacent vertebrae may comprise an L5 vertebra and an Si vertebra, two lumbar vertebrae, two sacral vertebrae, two thoracic vertebrae, or other two adjacent vertebrae of the same spine section or different spine sections. The access tool may include a cannula and stylet assembly, with the stylet extending through a lumen of the cannula.

The method may include inserting the access tool within an inner cancellous bone region of a vertebral body of the other one of the two adjacent vertebrae while avoiding contact with the one or more hardware components and inserting the neuromodulation device through the lumen of the access tool until at least a distal end of the neuromodulation device extends beyond the access tool within the inner cancellous bone region of the vertebral body of the other one of the two adjacent vertebrae. The method may alternative include inserting the access tool within an inner cancellous bone region of a vertebral body of a vertebra other than the two adjacent vertebrae and inserting the neuromodulation device through the lumen of the access tool until at least a distal end of the neuromodulation device extends beyond the access tool within the inner cancellous hone region of the vertebral body of the vertebra other than the two adjacent vertebrae.

The method may further include confirming modulation (e.g., ablation, denervation) of the basivertebral nerve. The neuromodulation device may comprise a radiofrequency probe (e.g., bipolar radiofrequency probe) configured to deliver radiofrequency energy sufficient to modulate (e.g., ablate, denervate) the nerve. The method may include confirming electrical isolation between electrodes of a bipolar radiofrequency probe and the one or more hardware components. The neuromodulation device may be configured to apply ultrasound energy sufficient to modulate (e.g., ablate, denervate) basivertebral nerve. The neuromodulation device may be configured to generate thermal energy sufficient to ablate the basivertebral nerve. In some embodiments, the neuromodulation device is a cryoablation device, a resistive heating device, or a laser device. In some embodiments, the neuromodulation device is configured to deliver a fluid configured to denervate the basivertebral nerve. In accordance with several embodiments, the neuromodulation device is non-implantable and only temporarily inserted during the modulation.

In some embodiments, modulating the basivertebral nerve comprises (i) stimulating the basivertebral nerve without significantly stimulating other tissue or (ii) denervating the basivertebral nerve with no electrical stimulation. In some embodiments, modulating the basivertebral nerve comprises (i) denervating only the basivertebral nerve or (ii) denervating the basivertebral nerve and neuromodulating other nerves, such as adjacent nerves to the basivertebral nerve.

The method may further include performing a total disc replacement between the adjacent vertebrae. In some embodiments, the step of performing a spinal fusion. procedure is replaced with performing a total disc replacement between the adjacent vertebrae.

In accordance with several embodiments, a method of treating low back pain of a patient includes inserting an access tool or assembly (e.g., cannula. and stylet assembly, with the stylet extending through a lumen of the cannula) within an inner cancellous bone region of a vertebral body of one of two adjacent vertebrae that have been fused together during a spinal fusion procedure (which may have been previously performed or instructed by a different entity or the same entity). The step of inserting includes avoiding contact with one or more hardware components left within or surrounding each of the adjacent vertebrae following the fusion procedure. The method also includes inserting a nerve modulation device (e.g., nerve ablation device, denervation device) through a lumen of the access tool or assembly (e.g., lumen of the cannula) until at least a distal end of the nerve modulation device (e.g., nerve ablation device) extends beyond the access tool within the inner cancellous bone region of the vertebral body. The method also includes modulating (e.g., ablating, denervating) a basivertebral nerve within the inner cancellous bone region using the nerve modulation device (e.g., bipolar radiofrequency probe, chemical ablation device, cryoablation device, microwave ablation device, ultrasound ablation device, laser ablation device). The method further includes removing the nerve modulation device and the access tool or assembly from the patient (e.g., the nerve modulation device is not implanted for modulation over time).

The step of inserting the access tool within the inner cancellous bone region may include extrapedicularly inserting the access tool or assembly within the inner cancellous bone region using an access location superior to a pedicle of a superior vertebra of the adjacent vertebrae. The step of inserting. the access tool within the inner cancellous bone region may alternatively or additionally include extrapedicularly inserting. the access tool within the inner cancellous bone region using an access location inferior to a pedicle of an inferior vertebra of the adjacent vertebrae.

The method may further include identifying the two adjacent vertebrae by determining that the two adjacent vertebrae exhibit at least one of: vertebral endplate defects, vertebral endplate degeneration, Modic changes or pre-Modic changes likely to result in a diagnosis of Modic changes in the future. The determining may be performed using magnetic resonance images (e.g., using [DEAL. MRI sequences) obtained of at least portions of the two adjacent vertebrae. The determining may also include use of artificial intelligence techniques that involve trained neural networks or deep learning models to facilitate the determination.

The two adjacent vertebrae may comprise an L5 vertebra and an SI vertebra, two adjacent lumbar vertebrae, or two adjacent sacral vertebrae. The two adjacent vertebrae may be vertebrae of a same spine section, level or segment or of different spine sections, levels or segments.

The modulation of the basivertebral nerve may include ablating the basivertebral nerve by applying, or causing to be delivered, thermal energy having a temperature between 70 and 90 degrees Celsius with a temperature ramp of between 1 and 3 degrees Celsius per second for a duration of between 5 minutes and 15 minutes.

The method may further include inserting the access tool or assembly within an inner cancellous bone region of a vertebral body of the other one of the two adjacent vertebrae while avoiding contact (e.g., not touching, coupling, or connecting) with the one or more hardware components and inserting the nerve ablation device through the lumen of the access tool until at least a distal end of the nerve ablation device extends beyond the access tool within the inner cancellous bone region of the vertebral body of the other one of the two adjacent vertebrae.

The method may also include confirming modulation (e.g., ablation) of the basivertebral nerve. For implementations in which the nerve modulation device is a bipolar radiofrequency probe, the method may include confirming electrical isolation between electrodes of the bipolar radiofrequency probe and the one or more hardware components.

In accordance with several embodiments, a kit for treating multiple independent sources of low back pain of a patient during a single therapeutic intervention includes a bone access assembly including an introducer cannula and a sharp stylet adapted to penetrate cortical bone, an intervertebral fusion assembly including multiple screws and multiple rods or plates, and a neuromodulation device configured to modulate (e.g., ablate, denervate) an intraosseous nerve (e.g., basivertebral nerve) within a vertebral body. In some embodiments, the neuromodulation device is non-implantable and is removed during the single therapeutic intervention.

In some embodiments, the intervertebral fusion assembly further includes graft material and/or a cage. The intervertebral fusion assembly may also include one or more implantable pharmacological agents configured to facilitate fusion, reduce inflammation, and/or reduce scar formation. The neuromodulation device may comprise a radiofrequency energy delivery probe configured to deliver radiofrequency energy sufficient to ablate the intraosseous nerve, The neuromodulation device may comprise a microwave energy delivery device, an ultrasound energy delivery device, a laser energy delivery device, a fluid delivery device, and/or a cryogenic cooling device. The kit may further include a nerve finding device or nerve monitoring device.

In accordance with several embodiments, a system for treating multiple independent sources of low back pain (e.g., chronic low back pain) of a patient during a single therapeutic intervention includes an introducer cannula and a sharp stylet adapted to be inserted through the introducer cannula and advanced together with the introducer cannula to penetrate cortical bone of a vertebral body. The sharp stylet is configured to be removed from the introducer cannula to facilitate insertion of other instruments within the vertebral body. The system also includes a plurality of fusion rods or plates, a plurality of bone screws adapted to anchor the plurality of fusion rods or plates to bone, and a radiofrequency generator. The system further includes a radiofrequency energy delivery probe configured to (i) be connected to the radiofrequency generator, (ii) be advanced through the introducer cannula, and (iii) apply energy from the radiofrequency generator in an amount sufficient to denervate a basivertebral nerve within the vertebral body. The system may further include an implantable pharmacological agent configured to facilitate fusion, reduce inflammation, and/or reduce scar formation.

In accordance with several embodiments, a system for treating multiple independent sources of low back pain of a patient during a single therapeutic intervention includes an introducer cannula and a sharp stylet adapted to be inserted through the introducer cannula and advanced together with the introducer cannula to penetrate cortical bone of a vertebral body. The sharp stylet is configured to be removed from the introducer cannula to facilitate insertion of other instruments within the vertebral body. The system also includes a plurality of fusion rods or plates, a plurality of bone screws adapted to anchor the plurality of fusion rods or plates to bone, and a neuromodulation device configured to modulate an intraosseous nerve within the vertebral body. The system may also include an implantable pharmacological agent configured to facilitate fusion, reduce inflammation, and/or reduce scar formation. The neuromodulation device may comprise a radiofrequency energy delivery probe configured to deliver radiofrequency energy sufficient to ablate the intraosseous nerve, a microwave energy delivery device, an ultrasound energy delivery device, a laser energy delivery device, a fluid delivery device, and/or a cryogenic cooling device. The system may include nerve finding device or nerve monitoring device.

Several embodiments of the invention have one or more of the following advantages: (i) increased patient satisfaction; (ii) increased efficiency; (iii) synergistic results; (iv) “one-and-done” procedure that does not require further surgical intervention; (v) reduced pain scores; (vi) reduced need for, or dependence on, opioids; (vii) simple adoption of a combined procedure performed by a single clinician (e.g., spine surgeon) without requiring extensive training; and/or (viii) treatment of multiple, independent sources of chronic low back pain. By reducing or eliminating the need for long-term pain management drugs in some embodiments, such as opioids, addiction to such pharmaceuticals may in turn be reduced.

For purposes of summarizing the disclosure, certain aspects, advantages, and novel features of embodiments of the disclosure have been described herein. It is to be understood that not necessarily all such advantages may be achieved in accordance with any particular embodiment of the disclosure provided herein. Thus, the embodiments disclosed herein may be embodied or carried out in a manner that achieves or optimizes one advantage or group of advantages as taught or suggested herein without necessarily achieving other advantages as may be taught or suggested herein.

The methods summarized above and set forth in further detail below describe certain actions taken by a practitioner; however, it should be understood that they can also include the instruction of those actions by another party. Thus, actions such as “performing a neuromodulation procedure” include “instructing the performance of a neuromodulation procedure.” Further aspects of embodiments of the disclosure will be discussed in the following portions of the specification. With respect to the drawings, elements from one figure may be combined with elements from the other figures.

### BRIEF DESCRIPTION OF THE DRAWINGS

Several embodiments of the disclosure will be more fully understood by reference to the following drawings which are for illustrative purposes only:

FIG. 1 illustrates various vertebral levels and vertebrae that may be treated by the systems and methods described herein.

FIGS. 2A, 2B and 2C illustrate various views of a portion of a patient's spine after fusion has been performed between two adjacent vertebrae. The figures show the residual hardware left in place following the fusion.

FIGS. 3A and 3B illustrate example methods of accessing at least one of the fused vertebrae following a fusion.

FIG. 4 illustrates an example system of access tools and a treatment device configured to access and modulate a nerve within a vertebral body.

FIGS. 5A and 5B are example flow charts of combined fusion and neuromodulation (e.g., denervation) procedures.

### DETAILED DESCRIPTION

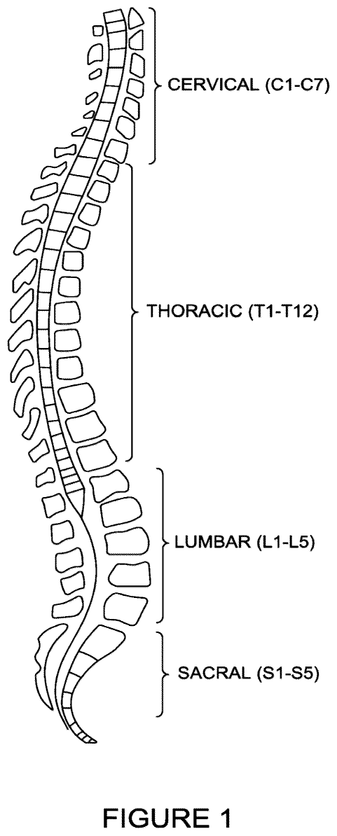

Several implementations described herein are directed to systems and methods for modulating nerves within or adjacent (e.g., surrounding) bone prior to, contemporaneous with, or following (post facto) one or more spinal fusion (e.g., interbody fusion), stabilization, and/or disc replacement (e.g., total disc replacement) operations. In some implementations, one or more intraosseous nerves (e.g., basivertebral nerve) within one or more bones (e.g., vertebral body, facet, lamina, pedicle) of the spine is modulated (e.g., ablated) for treatment, or prevention of, chronic back pain. The vertebral body/bodies may be located in any level of the vertebral column (e.g., cervical, thoracic, lumbar and/or sacral). FIG. 1 schematically illustrates a vertebral column and the various vertebral segments or levels that may be treated.

Multiple vertebral bodies may be treated in a single visit or combined therapeutic procedure (simultaneously or sequentially). The multiple vertebral bodies may be adjacent fused vertebral bodies and/or vertebral bodies adjacent the fused vertebral bodies. The multiple vertebral bodies may be located in a single spine segment (e.g., two adjacent vertebral bodies in the sacral spine segment (e.g., Si and S2) or lumbar spine segment (e.g., L3, L4 and/or L5)) or in different spine segments (e.g., an L5 vertebra in the lumbar spine segment and an Si vertebra in the sacral spine segment). The combined therapeutic procedure may be performed on vertebral levels or segments that are difficult to access via a percutaneous approach. All or portions of the procedure may be performed using an open surgical approach, a percutaneous approach, or an endoscopic (e.g., laparoscopic) approach.

Although spinal fusion (e.g., interbody fusion) is generally intended to reduce pain (e.g., chronic low back pain) caused by degenerative intervertebral discs, vertebral fractures, scoliosis, stenosis, or other spinal issues, spinal fusion procedures may actually result in additional pain. The additional pain may be caused by, or result from, the hardware, or instrumentation, used to accomplish the fusion (e.g., plates, screws, rods, cages) and/or the graft material autograft, allograft, bone graft substitutes, and/or bone morphogenetic proteins). For example, the hardware may change the biomechanics of the bone and result in offsetting of the load to different portions of the spine or vertebrae (e.g., due to loss of bending moments). Bone remodeling around screws of the hardware may also offset the load in the spine, resulting in increased pain. The pain may include persistent pain at the bone graft site, pain to vertebrae adjacent the fused vertebrae resulting from additional stress or loading on the bones adjacent to the fused vertebrae, and/or pain caused by damage to nerves in or surrounding the vertebral body by the hardware. The interbody graft material positioned between, within, or surrounding the vertebral bodies may exacerbate endplate pain as a result of increased loading and stress. The fusion procedure may include scraping and bloodying of one or more of the endplates (for example, if allograft material is used), thereby causing increased or additional endplate pain.

The fused vertebrae or vertebrae adjacent or surrounding the fused vertebrae may result in Modic changes (or symptoms associated therewith) caused by the fusion, or the fused vertebrae or adjacent vertebrae may have had pre-existing Modic changes (or symptoms associated therewith) prior to the fusion procedure. The Modic changes include pathological changes in a body of a vertebra and/or in an endplate of the vertebra or intervertebral disc. These Modic changes typically correlate to low back pain. The Modic changes may be identified using magnetic resonance imaging or other imaging modalities (e.g., CT, X-ray, fluoroscopic imaging). The Modic changes may include Type 1 Modic changes that include findings of inflammation and edema, type 2 Modic changes that include changes in bone marrow and increased visceral fat content, and/or type 3 Modic changes that include factures of trabecular bone of the vertebra and/or trabecular shortening and widening. In addition to Modic changes, the vertebrae may be identified as having pre-Modic changes prior to an official characterization of a Modic change or may be identified as having vertebral endplate degeneration or vertebral endplate defects (e.g., corner defects, erosive defects, focal defects, rim defects, etc.).

Neuromodulation may be performed prior to, contemporaneously with, or following vertebral fusion to prevent or treat pain that has been or may be generated by vertebral fusion and/or to treat pain caused by pre-existing Modic changes. For example, the neuromodulation may advantageously address the above-described pain caused by the fusion. If a fusion procedure is performed but fusion never really occurs due to a failure to fuse (e.g., pseudoarthrosis) or stabilize, then Modic changes or vertebral endplate degeneration or defects could be generated by the failed fusion that cause pain. In addition, the source of pain intended to be treated by spinal fusion (e.g., back pain caused by decompression or instability or tension or slippage) may be different than, or independent from, the source of pain intended to be treated by neuromodulation of a basivertebral nerve (e.g., pain caused by degradation or degeneration of endplates). Accordingly, performing either spinal fusion or neuromodulation (e.g., of basivertebral nerve) in isolation may not adequately address all sources of pain and may therefore provide incomplete treatment and reduced patient satisfaction. However, performing both fusion and neuromodulation (including performing them both in a single combined therapeutic procedure) may advantageously address both or all independent sources of chronic low back pain, thereby increasing patient comfort and satisfaction.

The neuromodulation may target intraosseous nerves (e.g., a basivertebral nerve) within one or more vertebral bodies. In some implementations, the one or more nerves being modulated are extraosseous nerves located outside the vertebral body or other bone (e.g., at locations before the nerves enter into, or after they exit from, the vertebral foramen). For example, nerve endings in endplates may be ablated in combination with total disc replacement or fusion procedures instead of ablating nerves within a vertebral body (e.g., when preparing for fusion using one or more spatulas). Portions of nerves within or on one or more vertebral endplates or intervertebral discs between adjacent vertebral bodies may be modulated (e.g., denervated, ablated).

[The combined fusion (and/or total disc replacement) and modulation of nerves or other tissue (e.g., basivertebral nerve denervation or ablation) may be performed to treat one or more indications, including but not limited to chronic low back pain, upper back pain, acute back pain, joint pain, tumors in the bone, and/or bone fractures. The combined therapeutic procedure may advantageously provide synergistic effects or complete all-in-one, “one-and-done” treatment that will not require further surgical or minimally invasive interventions, thereby increasing efficiency, reducing patient inconvenience (in terms of both time and pain), arid increasing patient satisfaction. As discussed previously, there may be multiple independent sources of back pain (e.g., (a) back pain caused by slipped discs, pinched spinal nerves, herniated discs, spinal stenosis, or spinal structural abnormalities and/or (b) back pain stemming from vertebral endplate degradation, such as caused by Modic changes or vertebral endplate degeneration or defects). Performing fusion or basivertebral nerve ablation alone may not treat or relieve all of the independent sources of a patient's chronic back pain. However, performing both fusion and basivertebral nerve ablation may advantageously address multiple or all independent sources of the patient's chronic back pain, thereby providing a total or more complete “one-and-done” treatment that provides total or more complete relief to the patient. The combined therapeutic procedure may advantageously result in better long-term back pain scores from patients (e.g., lower ODI scores, lower Oswestry low back pain scores, lower Roland-Morris disability scores, lower Quebec back pain disability scores, improved Visual Analogue scores) than would be achieved by performing either fusion or neuromodulation (e.g., basivertebral nerve ablation) in isolation.

In some embodiments, implantable pharmacological therapies and/or biologics are administered along with the combined therapies described herein. For example, these include pain management drugs delivered to reduce dependence, stem cells, cytokines, antibodies, growth factors, or other biologics that aid in the fusion process and/or reduce inflammation or scar tissue. In some embodiments, the neuromodulation therapies disclosed herein may be implantable on a short-term or long-term basis. In several embodiments, neuromodulation comprises or consists essentially of non-implantable denervation of the BVN (with no other nerves denervated and/or no stimulation). Alternatively, in one embodiment, the BVN is neuromodulated along with other nerves. In one embodiment, when two, three or more nerves are neuromodulated, the neuromodulation can be the same (e.g., all denervation) or different (e.g., a combination of denervation or stimulation). In several embodiments, no stimulation (e.g., electrical stimulation) is provided (e.g., at the time of the combined procedures and/or to facilitate the fusion).

In some implementations, fractures within the bone may be treated in addition to neuromodulation treatment and/or ablation of tumors by applying heat or energy and/or delivering agents or bone filler material to the bone. For example, bone morphogenetic proteins and/or bone cement may be delivered in conjunction with vertebroplasty or other procedures to treat fractures or promote bone growth or bone healing. In some implementations, energy is applied using the neuromodulation tools and then agents and/or bone filler material is delivered in a combined procedure. In some aspects, vertebral compression fractures (which may be caused by osteoporosis or cancer) are treated in conjunction with energy delivery to modulate nerves and/or cancerous tissue to treat potential additional sources of back pain.

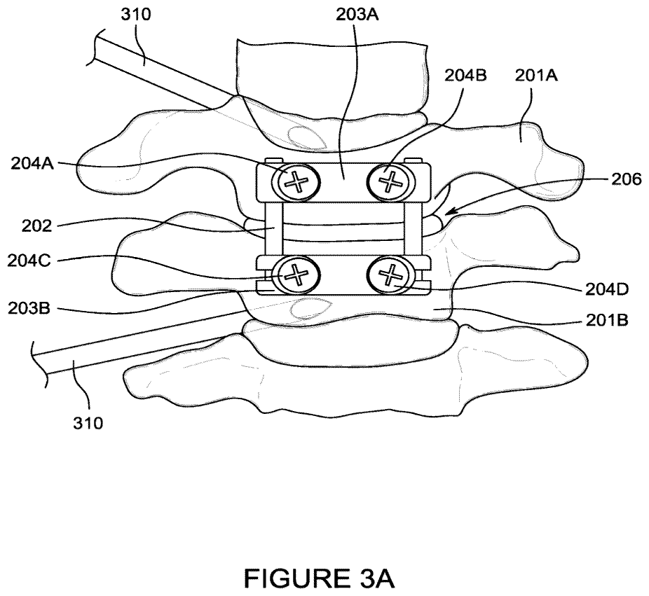

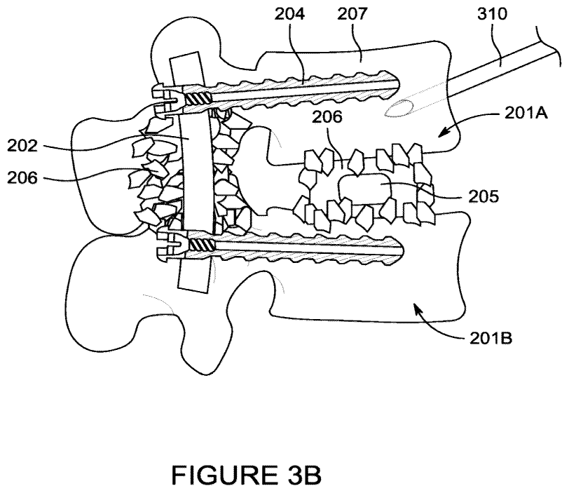

respectively illustrate a posterior view and a side view (with a midline cutaway) of two fused vertebrae 201A, 201B (collectively 201) after fusion or stabilization has been performed. As shown, fusion of the vertebrae 201 may involve use of rods 202 and plates 203 (shown in FIG. 3A) that are fixed to the adjacent vertebrae 201A, 201B by screws 204. The fusion may also include insertion of a cage 205 and/or graft material 206 (e.g., mechanical inserts, bone growth substances or fillers) between the adjacent vertebrae 201 after removal of some or all of the intervertebral disc. The graft material 206 may include, for example, autograft, allograft, bone graft substitutes, and/or bone morphogenetic proteins. The fusion may include scraping and bloodying of endplate surfaces, especially if allograft is used, in order to promote bone growth.

In some implementations, the screws 204 are inserted through a pedicle or other bony portion of the spine and into an inner cancellous bone region 207 of the vertebrae 201. The screws 204 may be used to stabilize a portion of the spine (e.g., one or more spine segments or levels) or to repair spinal defects (e.g., scoliosis, herniated discs, fractures, etc.). The type of spinal fusion may comprise anterior lumbar interbody fusion (ALIF), posterior lumbar interbody fusion CALIF), lateral lumbar interbody fusion (LLIF), posterolateral gutter fusion, extreme lateral interbody fusion (XLEF) and/or transforaminal lumbar interbody fusion (11JF). The screws 204 may be pedicle screws.

If neuromodulation (e.g., basivertebral nerve ablation within one or more vertebral bodies) is performed prior to fusion, any method of access (e.g., transpedicular, extrapedicular and/or anterior access) may be used, which may result in a less complex and less difficult overall combined therapeutic procedure in some embodiments. The fusion could then be performed using the same or different method of access as the access used for the neuromodulation procedure. For example, if the fusion desired or needed to be performed is an ALIF type of fusion, transpedicular access may not be used. For neuromodulation performed following vertebral fusion, the residual hardware or instrumentation that remains in and surrounding the adjacent vertebral bodies may make it more difficult for clinicians to access a desired target region of an intraosseous nerve (e.g., basivertebral nerve) modulation (e.g., denervation, ablation) therapy within one or more vertebral bodies.

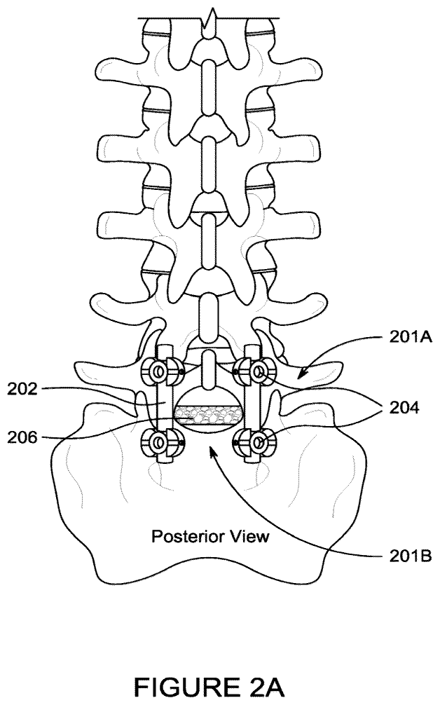

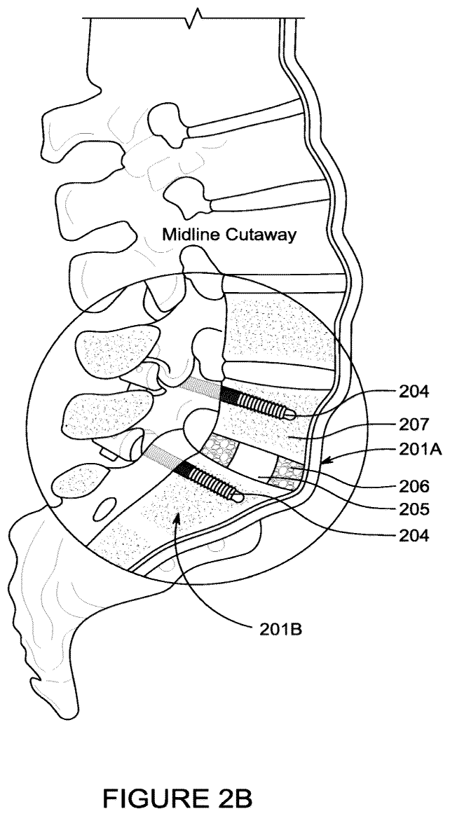

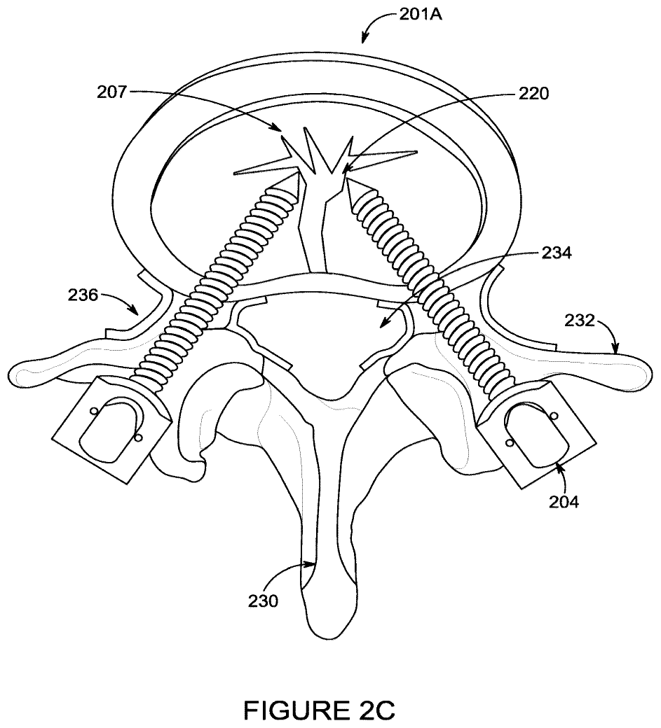

FIG. 2C illustrates a top cross-section view of one of the vertebral segments or levels. The vertebral segment includes a vertebral body 201A, a spinous process 230, transverse processes 232, a spinal canal 234, and pedicles 236. The vertebral body 201A includes an inner cancellous bone region 207 having a basivertebral nerve trunk 220. The vertebral body 201A includes hardware (e.g., screws 204) having been inserted through the pedicles 236. A desired target region for ablation of a basivertebral nerve trunk 220 may be, for example, 0.5 cm to 3 cm (e.g., 1 cm to 3 cm) anterior of a posterior border of the vertebral body 201A and generally at a lateral-lateral midline of the vertebral body 201A and generally aligned with the spinous process 230 of the vertebra corresponding to the vertebral body 201A.

FIGS. 3A and 3B illustrate example methods of accessing an inner cancellous bone region 207 of one of the fused vertebrae 201 following the fusion procedure. FIG. 3A is a side view of a posterior side of a spine and FIG. 3B is a lateral side view of the spine. Various methods of access may be used to access the inner cancellous bone region 207 where intraosseous nerves (e.g., basivertebral nerves) within the vertebral body are located to perform the neuromodulation (e.g., ablation, denervation).

In some implementations, as shown in FIG. 3A, the inner cancellous bone region 207 is accessed extrapedicularly (e.g., without traversing through a pedicle). For example, transpedicular access may not be possible because the residual hardware (e.g., pedicle screws 204) is impeding transpedicular access. FIG. 3A schematically illustrates two different extrapedicular access paths by an access tool/assembly 310. In some implementations, the access path may involve insertion of an access tool/assembly 310 superior to (e.g., above) a pedicle and arcing over residual hardware (e.g., screws 204, rods 202, plates 203) left in place from the fusion procedure. In some implementations, the access path may involve insertion of the access tool/assembly 310 inferior to (e.g., below) a pedicle and arcing under residual hardware (e.g., screws, rods, plates) left in place from the fusion procedure. For extrapedicular access of the superior vertebra (e.g., L5 vertebra) of the two fused vertebrae, the access tool 310 (e.g., introducer cannula/stylet assembly) may be inserted superior to the plate 203A and the screws 204A, 204B (as shown in FIG. 3A). For extrapedicular access of the inferior vertebra (e.g., S1 vertebra) of the two fused vertebrae, the access tool 310 (e.g., introducer cannula/stylet assembly) may be inserted inferior to the plate 203B and the screws 204C, 20413 (as also shown in FIG. 3A).

The access tool/assembly 310 may advantageously be inserted and advanced along an access path that avoids contact with a spinal nerve exiting from an adjacent level of the spine. In accordance with several embodiments, an access path below inferior to) the pedicle may be used because contact with a spinal nerve is less likely if access is below (inferior to) the pedicle and/or because this access path will likely get the treatment device for neuromodulation closer to the midline of the vertebral body Where the target treatment zone (e.g., basivertebral nerve trunk 220) is likely to be located for the neuromodulation. In addition, neural monitoring (e.g., using nerve finding or nerve monitoring tools and techniques) may be performed to avoid contact with the spinal nerve upon extrapedicular access following fusion. The nerve monitoring tools and techniques may incorporate measurements of electromyography and somatosensory evoked potentials.

In some implementations, the inner cancellous bone region 207 is accessed transpedicularly (through a pedicle). For example, transpedicular access for the neuromodulation therapy may be used if a unilateral fusion has been performed, such that contralateral transpedicular access through the other pedicle remains available. Transpedicular access may also be possible if an anterior fusion (ALIT) has been performed previously. However, transpedicular access may not be possible in some instances due to location of one or more of the screws 204, which may be inserted through one or more of the pedicles. In some implementations, as shown in FIG. 3B, an anterior approach is used to access the inner cancellous bone region 207 with the access tool/assembly 310 if a transpedicular approach is not available. In some implementations (not shown), the inner cancellous bone region 207 is accessed using an extreme lateral approach or a transforaminal approach, such as used in XLIF or TLIF interbody fusion procedures.

Access to the inner cancellous bone region may be performed under an imaging modality in order to prevent interference or contact with the fusion hardware, or instrumentation (e.g., screws 204, rods 202, plates 203). The imaging modality may advantageously provide real-time imaging or access may be based on previously-obtained images. The imaging modality may include fluoroscopy, computed tomography, magnetic resonance imaging, ultrasound, X-ray, and/or the like. Access may also be performed using an automated system (e.g., robotic guidance system), which may incorporate an imaging modality, virtual or augmented reality technology, and/or artificial intelligence data or techniques, to facilitate more precise insertion and positioning of the access tools.

Access tools may include an introducer assembly including an outer cannula and a sharpened stylet, an inner cannula configured to be introduced through the outer cannula, and/or one or more additional stylets, curettes, or drills to facilitate access to an intraosseous location within a vertebral body or other bone. The access tools (e.g., outer cannula, inner cannula, stylets, curettes, drills) may have pre-curved distal end portions or may be steerable, deflectable, flexible, or curveable. Any of the access tools may be drill-tipped or beveled or they may have blunt or rounded, atraumatic distal tips. Curved drills may be used to facilitate formation of curved access paths within bone. Any of the access tools may be advanced over a guidewire in some implementations.

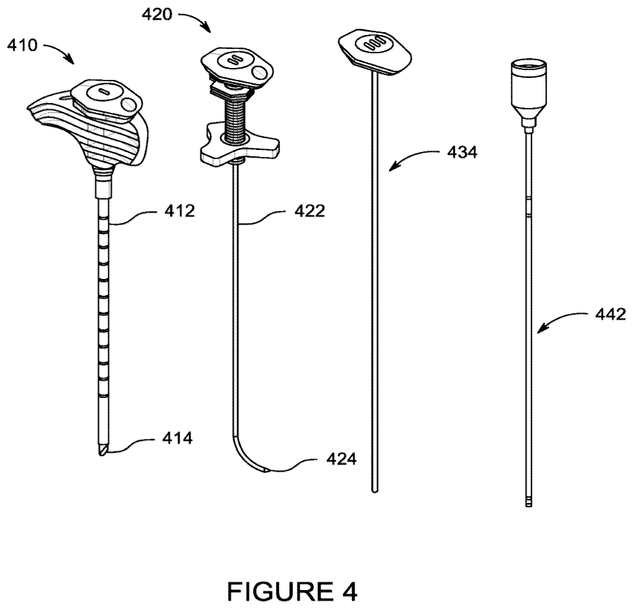

With reference to FIG. 4, in one implementation, the access tools for neuromodulation therapy include an introducer assembly 410 comprised of an introducer cannula 412 and an introducer stylet 414, a curved cannula assembly 420 comprised of a curved cannula 422 and a J-stylet 424, and a straight stylet 434. The introducer stylet 414 may be bevel tipped or trocar tipped. The introducer stylet 414 is configured to be received in a lumen of the introducer cannula 412 such that a distal tip of the introducer stylet 414 protrudes from an open distal tip of the introducer cannula 412, thereby forming the introducer assembly 410 in combination. The i-stylet 424 is configured to be received in a lumen of the curved cannula 422 such that a distal tip of the J-stylet 424 protrudes from an open distal tip of the curved cannula. 422, thereby forming the curved cannula assembly 420 in combination. The curved cannula 422 and the J-stylet 424 may each comprise a straight proximal main body portion and a curved distal end portion. The curves of the curved distal end portions of the curved cannula 422 and the j-stylet 424 correspond to each other. The straight stylet 434 is a flexible channeling stylet and may include an embedded radiopaque marker band in a distal end portion of the stylet to facilitate visualization under fluoroscopic or CT imaging. The access tools may be provided as a kit that may optionally additionally include one or more additional straight introducer cannulas, curved cannulas (e.g., having a curved distal end portion of a different curvature than the first curved cannula), drills, and/or an additional straight stylet (e.g., having a different length than the first straight stylet}. Some kits may include at least two of every access instrument and at least two treatment devices. The optional additional access tools may be adapted to access one or more additional vertebrae in the same spinal segment or a different spinal segment.

At least some of the access tools may be formed of a variety of flexible materials (e.g., ethylene vinyl acetate, polyethylene, polyethylene-based polyolefin elastomers, polypropylene, polypropylene-based elastomers, styrene butadiene copolymers, thermoplastic polyester elastomers, thermoplastic polyurethane elastomers, thermoplastic vulcanizate polymers, and/or the like). ‘The access tools may include chevron designs or patterns or slits along the distal end portions to increase flexibility or bendability. Any of the access tools may be manually or automatically rotated to facilitate a desired trajectory. Other access tools (e.g., introducer assembly 410 and straight stylets) may be formed of rigid materials. Portions of an access tool may be flexible while other portions are rigid.

In some implementations, an outer cannula assembly (e.g., introducer assembly) includes a straight outer cannula and a straight stylet configured to be received within the outer cannula. The outer cannula assembly (e.g., introducer assembly 410) may be inserted first to penetrate an outer cortical shell of a bone and provide a conduit for further access tools to the inner cancellous bone. An inner cannula assembly (e.g., curved cannula assembly 420) may include a cannula having a pre-curved or steerable distal end portion and a stylet having a corresponding pre-curved or steerable distal end portion. Multiple stylets having distal end portions with different curvatures may be provided in a kit and selected from by a clinician. The inner cannula assembly may alternatively be configured to remain straight and non-curved.

The treatment devices (e.g., treatment probes) may be any device capable of modulating tissue (e.g., nerves, tumors, bone tissue). Any energy delivery device capable of delivering energy can be used (e.g., RF energy delivery devices, microwave energy delivery devices, laser devices, infrared energy devices, other electromagnetic energy delivery devices, ultrasound energy delivery devices, and the like). FIG. 4 includes one embodiment of an RF energy delivery device 442. The RF energy delivery device 442 includes a bipolar pair of electrodes at a distal end portion of the device. The bipolar pair of electrodes includes an active tip electrode and a return ring electrode spaced apart from the active tip electrode. The RF energy delivery device 442 may include one or more temperature sensors (e.g., thermocouples, thermistors) positioned on an external surface of or embedded within a shaft of the energy delivery device. The RF energy delivery device 442 may not employ internally circulating cooling, in accordance with several implementations.

In accordance with several implementations, thermal energy may be applied within a cancellous bone portion (e.g., by one or more radiofrequency (RF) energy delivery instruments coupled to one or more RF generators). The thermal energy may be conducted by heat transfer to the surrounding cancellous bone, thereby heating up the cancellous bone. In accordance with several implementations, the thermal energy is applied within a specific frequency range and having a sufficient temperature and over a sufficient duration of time to heat the cancellous bone such that the basivertebral nerve extending through the cancellous bone of the vertebral body is modulated. In several implementations, modulation comprises permanent ablation or denervation or cellular poration (e.g., electroporation). In some implementations, modulation comprises temporary denervation or inhibition. In some implementations, modulation comprises denervation without necrosis of tissue.

For thermal energy, temperatures of the thermal energy may range from about 70 to about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp may range from 0.1-5 degrees Celsius/second (e.g., 0.5-2.0 degrees Celsius/second, 1.0-3.0 degrees Celsius/second, 1.5-4.0 degree Celsius/second, 2.0-5.0 degrees Celsius/second). The time of treatment may range from about 10 seconds to about 1 hour (e.g., from 10 seconds to 1 minute, 1 minute to 5 minutes, from 5 minutes to 15 minutes, from 5 minutes to 10 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy may be delivered as an alternative to or in sequence with continuous energy. For radiofrequency energy, the energy applied may range from 350 kHz to 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz lie 5 kHz, 475 kHz d: 5 kHz, 487 kHz 5 kHz). A power of the radiofrequency energy may range from 5 W to 30 W (e.g., from 5 W to 15 W, from 5 W to 20 W, from 8 W to 12 W, from 10 W to 25 4V, from 15 W to 25 W, from 20 W to 30 W, from 8 W to 24 W, and overlapping ranges thereof, or any value within the recited ranges). In accordance with several implementations, a thermal treatment dose (e.g., using a cumulative equivalent minutes (Cl M) 43 degrees Celsius model or a comparable Arrhenius model) is between 200 and 300 CEM (e.g., between 200 and 240 CEM, between 230 CEM and 260 CEM, between 240 CEM and 280 CEM, between 260 CEM and 300 CEM) or greater than a predetermined threshold (e.g., greater than 240 CEM).

In some embodiments, the neuromodulation device comprises one or more microwave antennae or elements configured to deliver energy sufficient to modulate (e.g., ablate, denervate) nerves. The microwave antenna may be positioned along a distal end of the neuromodulation device. The microwave antenna may be located within a centering and/or cooling balloon. The neuromodulation device may be adapted to apply energy with the one or more microwave antennae directly in contact with the nerves or from a distance away from the nerves. The frequency of the microwave energy may range from 500 MHz to 20 GHz (e.g., 500 MHz to 900 MHz, 800 MHz to 1 GHz, 915 MHz, 1 GHz to 3 GHZ, 2.45 GHZ, 2.5 GHz to 4 GHz, 3 GHz to 8 GHz, 6 GHz to 10 GHz, 10 GHz to 20 GHZ, overlapping ranges thereof, or any value within the recited ranges). The microwave energy may advantageously ablate nervous tissue without ablating bone tissue. The neuromodulation device may include multiple antennas operating simultaneously.

In some embodiments, the neuromodulation device comprises one or more ultrasound transducers or elements configured to delivery energy sufficient to modulate (e.g., ablate, denervate) nerves. The one or more ultrasound transducers or elements may be positioned along a distal end of the neuromodulation device. The transducers may be adapted to deliver focused energy in a targeted direction as opposed to omnidirectionally. The one or more ultrasound transducers may be located within a centering and/or cooling balloon. The neuromodulation device may be adapted to apply energy with the one or more ultrasound transducers from a distance away from the nerves. The frequency of the ultrasound energy may range from 200 kHz to 50 MHz (e.g., 200 kHz to 900 kHz, 800 kHz to 1 MHz, 1 MHz to 3 MHz, 2.5 MHz to 4 MHz, 3 MHz to 8 MHz, 6 MHz to 10 MHz, 10 MHz to 20 MHz, 15 MHz to 40 MHz, overlapping ranges thereof, or any value within the recited ranges). The acoustic energy may advantageously ablate nervous tissue without ablating intervening bone tissue if delivered from outside the vertebral body or even from within the vertebral body. The neuromodulation device may include multiple ultrasound transducers operating simultaneously. The ultrasound transducer(s) may be configured to be used for ablation and/or diagnostic imaging.

Cooling may optionally be provided to prevent surrounding tissues from being heated during the nerve modulation procedure. The cooling fluid may be internally circulated through the delivery device from and to a fluid reservoir in a closed circuit manner (e.g., using an inflow lumen and an outflow lumen). The cooling fluid may comprise pure water or a saline solution having a temperature sufficient to cool electrodes (e.g., 2-10 degrees Celsius, 5-10 degrees Celsius, 5-15 degrees Celsius). The cooling fluid may comprise a gas, such as carbon dioxide. Cooling may be provided by the same instrument used to deliver thermal energy (e.g., heat) or a separate instrument. In accordance with several implementations, cooling is not used.

In some implementations, ablative cooling may be applied to the nerves or bone tissue instead of heat (e.g., for cryoneurolysis or cryoablation applications). The temperature and duration of the cooling may be sufficient to modulate intraosseous nerves (e.g., ablation, or localized freezing, due to excessive cooling). The cold temperatures may destroy the myelin coating or sheath surrounding the nerves. The cold temperatures may also advantageously reduce the sensation of pain. The cooling may be delivered using a hollow needle under fluoroscopy or other imaging modality.

In sonic implementations, one or more fluids or agents may be delivered to a target treatment site to modulate a nerve. The agents may comprise bone morphogenetic proteins, for example. In some implementations, the fluids or agents may comprise chemicals for modulating nerves (e.g., chemoablative agents, alcohols, phenols, nerve-inhibiting agents, or nerve stimulating agents). The fluids or agents may be delivered using a hollow needle or injection device under fluoroscopy or other imaging modality.

One or more treatment devices (e.g., probes) may be used simultaneously or sequentially. For example, the distal end portions of two treatment devices may be inserted to different locations within a vertebral body or other bone or within different vertebral bodies or bones. Radiofrequency treatment probes may include multiple electrodes configured to act as monopolar, or unipolar, electrodes or as pairs of bipolar electrodes. The treatment device(s) may also be pre-curved or curveable such that the curved stylet is not needed or may have sharp distal tips such that additional sharpened stylets are not needed. In some implementations, any or all of the access tools and the treatment devices are MR-compatible so as to be visualized under MR imaging.

In accordance with several embodiments, regardless of type of access (e.g., transpedicular, extrapedicular, anterior, etc.), the access tools (and especially the treatment device) are inserted and advanced along an access path such that the treatment delivery elements (e.g., electrodes) of the treatment devices are electrically isolated from (e.g., more than 1 mm away from) the residual fusion hardware when the treatment device is delivering treatment to a target treatment zone or site. The access tools (and especially the treatment device) may also be inserted and advanced along an access path such that the treatment delivery elements (e.g., electrodes) of the treatment devices are far enough away from (e.g., greater than 1 mm, greater than 1.5 mm, greater than 2 mm, greater than 2.5 mm, greater than 3 mm) the residual fusion hardware (e.g., pedicle screws) such that the residual fusion hardware is not acting as an appreciable thermal sink for the treatment devices. The electrical isolation and thermal insulation prevents the residual fusion hardware from conducting electrical current or thermal energy (e.g., heat) which could cause pain, discomfort, or undesired ablation of tissue outside the target treatment zone or site. In some embodiments, monitoring tools may be used that contact the residual fusion hardware (e.g., pedicle screws) to confirm or verify (using monitoring software or detection circuitry) electrical isolation of the residual fusion hardware from the energy delivery elements of the treatment device(s) following placement of the treatment device(s).

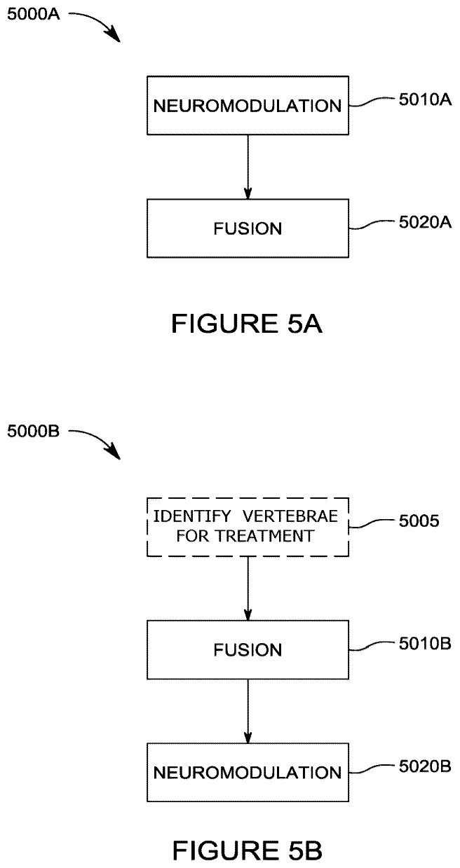

FIGS. 5A and 5B are example flow charts of methods of treatment involving combined fusion and neuromodulation (e.g., denervation) therapy. Again, the fusion and neuromodulation may be instructed by the same entity or by different entities. With reference to FIG. 5A., a process 5000A of treating pain (e.g., chronic low back pain) in a patient includes performing neuromodulation (Block 5010A) of nerves within or surrounding the body of one or more adjacent vertebrae. As discussed herein, the neuromodulation may include ablation of nerves using any of the access and neuromodulation. tools described herein (e.g., access and treatment tools described in connection with FIG. 4), as well as alternative access and neuromodulation tools. For example, the neuromodulation may include denervating (e.g., ablating) one or more intraosseous nerves (e.g., basivertebral nerve), such as the IN'FRACEPT® RF ablation procedure offered by applicant Relievant Medsystems, Inc., such as described in U.S. Pat. Nos. 8,419,730; 8,613,744; 9,724,151; and/or U.S. Pat. No. 8,361,067; the entire content of each of which is hereby incorporated herein by reference.

In one implementation, when preparing for fusion, a spatula that is to be used during the fusion surgery preparation could include resistive wire, thereby acting like a branding iron to heat the intraosseous nerves or bone, the vertebral endplates, and/or extraosseous nerves. In some implementations, a monopolar spatula or two bipolar spatulas may be used to ablate nerve endings prior to, or in conjunction with, a fusion procedure as a “belt-and-suspenders” approach. In some implementations, a balloon catheter with an ultrasound transducer can be used to ablate the endplate nerves since the endplate is cartilaginous and not bony. Other tissue in addition to, or alternative to, nerves may also be treated or otherwise affected (e.g., tumors or other cancerous tissue).

The method further includes performing a spinal fusion (Block 5020A) between two adjacent vertebrae of the patient (e.g., at least one of which received the neuromodulation therapy performed at Block 5010A). In some implementations, one or more pedicle screws may be inserted through transpedicular access channels formed by the access instruments during the neuromodulation therapy. All or a portion of the spinal fusion and/or neuromodulation may be performed using automated systems or techniques (e.g., robotic; assisted spine surgery techniques using a robotic guidance and/or surgical system and/or artificial intelligence systems, techniques and data). For example, pedicle screws may be inserted or implanted using a robotic-guided implantation system and technique. The procedures may be performed using an open surgical approach or a percutaneous approach. In some implementations, all or a portion of the spinal fusion and/or neuromodulation may be performed manually. In some implementations, total disc replacement (e.g., artificial disc replacement) may be performed instead of, or in. addition to, the spinal fusion. Total disc replacement and fusion procedures may incorporate use of 3D-printed components, such as cages, artificial discs, pedicle screws, plates, rods, and/or the like.

FIG. 5B illustrates an alternative process 5000E of treating pain (e.g., chronic low back pain) in a patient. The process 5000B optionally includes a step of identifying one or more vertebrae likely to be a source of chronic low back pain at the present time or in the future (Block 5005). For example, identification may include identification of Modic changes or pre-Modic changes associated with one or more vertebrae indicative of pain. The identification of i:Iodic changes may indicate which vertebrae to treat via the combined fusion and neuromodulation therapeutic procedure. The identification of Modic changes may be optional and need not be performed. ‘Vertebral endplate degeneration or endplate defects other than Modic changes may be identified, such as described above. Multiple different independent sources of pain may be identified as needing to be treated (e.g., pain originating from one or more intervertebral discs or stemming from one or more vertebral endplates or within one or more intraosseous locations within vertebrae). Intraosseous sources of pain could include vertebral fractures or tumors.

The process 5000B of FIG. 5B involves performing the neuromodulation and fusion in opposite order of the process 5000A of FIG. 5A. The process 5000B includes performing a spinal fusion (Block 5010B) between two adjacent vertebrae of the patient. The spinal fusion includes leaving one or more hardware components (e.g., screws, rods, plates) within or surrounding a body of each of the adjacent vertebra. In some implementations, total disc replacement (e.g., artificial disc replacement) may be performed instead of, or in addition to, the spinal fusion at Block 50109. The process 50009 then includes performing neuromodulation (Block 50209) within or surrounding the vertebral body of one or both of the adjacent vertebrae. For example, the neuromodulation may include denervating (e.g., ablating) one or more intraosseous nerves (e.g., basivertebral nerve), such as described in U.S. Pat. Nos. 8,419,730; 8,613,744; and/or U.S. Pat. No. 8,361,067; the entire content of each of which is hereby incorporated herein by reference.

In various implementations, the neuromodulation step includes inserting an access tool or assembly within an inner cancellous bone region of a vertebral body of one of the two adjacent vertebrae while avoiding contact (e.g., does not involve touching or coupling) with the one or more hardware components. The access tool may include an introducer comprising a lumen extending therethrough to facilitate access to the inner cancellous bone region of the vertebral body. The access tool may include a cannula and stylet assembly, with the stylet extending through a lumen of the cannula such that a sharp distal tip of the stylet extends beyond an open distal end of the cannula to facilitate penetration of bone tissue. The neuromodulation step may further include inserting a neuromodulation device through the lumen of the access tool until at least a distal end of the neuromodulation device extends beyond the access tool within the inner cancellous bone region of the vertebral body. The neuromodulation step may also include modulating a basivertebral nerve or other intraosseous nerves within the inner cancellous bone region using the neuromodulation device and removing the neuromodulation device and the access tool from the patient (e.g., the neuromodulation device is not implanted but is removed following the neuromodulation step). The neuromodulation step may additionally include modulating a nerve (e.g., basivertebral nerve) within the other one of the two adjacent vertebrae and/or modulating one or more nerves (e.g., basivertebral nerves or other intraosseous nerves) within one or more vertebrae other than the vertebrae that were fused.

The neuromodulation step may additionally or alternatively include modulating one or more extraosseous nerves (e.g., nerves of one or more endplates, nerves within a foramen (e.g., basivertebral foramen). For example, the basivertebral nerve may be ablated outside the vertebral body at a location of entry within the vertebral body if the residual hardware form the fusion is impeding access or is too close to a desired target ablation zone that could result in excessive heating of the screws or thermal sinking caused by the screws, thereby making denervation ineffective or less effective, or making the denervation take longer. For example, the neuromodulation step may be performed as described in applicant's U.S. Pat. No. 10,390,877, the entire content of which is hereby incorporated herein by reference.

The step of inserting the access tool within the inner cancellous bone region may include extrapedicularly inserting the access tool within the inner cancellous bone region using an access location superior to the one or more hardware components associated with a superior vertebra (e.g., L5 vertebra) of the adjacent vertebrae. The step of inserting the access tool within the inner cancellous bone region may additionally or alternatively include extrapedicularly inserting the access tool within the inner cancellous bone region using an access location inferior to the one or more hardware components associated with an inferior vertebra (e.g., SI vertebra) of the adjacent vertebrae.

The processes illustrated in FIGS. 5A and 5B may optionally include confirming modulation (e.g., ablation) of the basivertebral nerve or other nerve or tissue. The neuromodulation device may comprise a radiofrequency energy delivery device (e.g., bipolar RF probe) configured to deliver radiofrequency energy sufficient to modulate (e.g., ablate, denervate, stimulate) a nerve (e.g., basivertebral nerve). The neuromodulation device may comprise one or more ultrasound transducers configured to apply ultrasound energy sufficient to modulate a nerve. The neuromodulation device may be configured to generate thermal energy sufficient to modulate the basivertebral nerve. In some implementations, the neuromodulation device is a cryoablation device and/or is configured to deliver a fluid (e.g., ablative fluid, such as a chemical liquid or a gas such as water vapor or steam) sufficient to modulate the nerve. The neuromodulation device may be a resistive heating device or a laser device.

In some implementations, the system comprises various features that are present as single features (as opposed to multiple features). For example, in one embodiment, the system includes a single radiofrequency generator, a single introducer cannula with a single stylet, a single radiofrequency energy delivery device or probe, and a single bipolar pair of electrodes. A single thermocouple (or other means for measuring temperature) may also be included. Multiple features or components (e.g., multiple radiofrequency energy delivery devices or probes) are provided in alternate embodiments.

In some implementations, the system comprises one or more of the following: means for tissue modulation (e.g., an ablation or other type of modulation catheter or delivery device), means for monitoring temperature (e.g., thermocouple, thermistor, infrared sensor), means for imaging (e.g., stereotactic guidance, MRI, CT, fluoroscopy), means for accessing (e.g., introducer assembly, curved cannulas, drills, curettes), etc. In some implementations, the means may comprise automated (e.g., robotic) means to facilitate increased precision, quicker procedures, and decreased blood loss.

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying thermal energy” include “instructing the applying of thermal energy.”

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5., 90.5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 2:1” includes “2:1.” For example, the terms “approximately”, “about”, and “substantially” as used herein represent an amount close to the stated amount that still performs a desired function or achieves a desired result.

We claim:

1. A method of treating multiple independent sources of low back pain of a patient during a single therapeutic intervention, the method comprising:
performing a spinal fusion procedure between two adjacent vertebrae of the patient, including leaving one or more hardware components within or surrounding each of the adjacent vertebrae;
inserting an access tool within a cancellous bone region of a vertebral body of one of the two adjacent vertebrae, the access tool comprising a lumen extending therethrough to facilitate access to the cancellous bone region;
inserting a neuromodulation device through the lumen of the access tool until at least a distal end of the neuromodulation device extends beyond the access tool within the cancellous bone region;
modulating a basivertebral nerve within the cancellous bone region using the neuromodulation device; and
removing the neuromodulation device and the access tool from the patient.

2. The method of claim 1, wherein the step of inserting the access tool within the cancellous bone region comprises extrapedicularly inserting the access tool within the cancellous bone region using an access location superior to a pedicle of a superior vertebra of the adjacent vertebrae.

3. The method of claim 1, wherein the step of inserting the access tool within the cancellous bone region comprises extrapedicularly inserting the access tool within the cancellous bone region using an access location inferior to a pedicle of an inferior vertebra of the adjacent vertebrae.

4. The method of claim 1, further comprising identifying the two adjacent vertebrae by determining that the two adjacent vertebrae exhibit one or more Modic changes.

5. The method of claim 1, wherein the two adjacent vertebrae comprise an L5 vertebra and an S1 vertebra.

6. The method of claim 1, wherein the two adjacent vertebrae comprise lumbar vertebrae.

7. The method of claim 1, further comprising inserting the access tool within a cancellous bone region of a vertebral body of the other one of the two adjacent vertebrae while avoiding contact with the one or more hardware components and inserting the neuromodulation device through the lumen of the access tool until at least a distal end of the neuromodulation device extends beyond the access tool within the cancellous bone region of the vertebral body of the other one of the two adjacent vertebrae.

8. The method of claim 1, wherein the neuromodulation device is configured to deliver radiofrequency energy sufficient to ablate the basivertebral nerve.

9. The method of claim 1, wherein modulating the basivertebral nerve comprises ablating the basivertebral nerve with ultrasound energy.

10. A method of treating multiple independent sources of low back pain of a patient having two adjacent vertebrae in a lumbar or lumbosacral region, each vertebra comprising a vertebral body with a cortical bone region and a cancellous bone region containing intraosseous nerves, during a single therapeutic intervention, the method comprising:
performing a spinal fusion procedure between the two adjacent vertebrae of the patient, including leaving one or more hardware components within or surrounding the adjacent vertebrae;
inserting an access tool through the cortical bone region into the cancellous bone region of a vertebral body of one of the two adjacent vertebrae while avoiding contact with the one or more hardware components, the access tool comprising a lumen extending therethrough to facilitate access to the cancellous bone region;
inserting a neuromodulation device through the lumen of the access tool until at least a distal end of the neuromodulation device extends beyond the access tool and into the cancellous bone region; and
modulating an intraosseous nerve within the cancellous bone region using the neuromodulation device.

11. The method of claim 10, wherein inserting the access tool within the cancellous bone region comprises extrapedicularly inserting the access tool within the cancellous bone region using an access location superior to a pedicle of a superior vertebra of the adjacent vertebrae.

12. The method of claim 10, wherein inserting the access tool within the cancellous bone region comprises extrapedicularly inserting the access tool within the cancellous bone region using an access location inferior to a pedicle of an inferior vertebra of the adjacent vertebrae.

13. The method of claim 10, further comprising identifying the two adjacent vertebrae by determining that the two adjacent vertebrae exhibit one or more Modic changes.

14. The method of claim 10, wherein the two adjacent vertebrae comprise an L5 vertebra and an S1 vertebra.

15. The method of claim 14, wherein the two adjacent vertebrae comprise lumbar vertebrae.

16. The method of claim 11, further comprising inserting the access tool within a cancellous bone region of a vertebral body of the other one of the two adjacent vertebrae while avoiding contact with the one or more hardware components and inserting the neuromodulation device through the lumen of the access tool until at least a distal end of the neuromodulation device extends beyond the access tool and into the cancellous bone region of the vertebral body of the other one of the two adjacent vertebrae.

17. The method of claim 10, wherein the neuromodulation device is configured to deliver radiofrequency energy sufficient to ablate the intraosseous nerve.

18. The method of claim 17, wherein modulating the intraosseous nerve comprises ablating the intraosseous nerve.

19. The method of claim 10, wherein the intraosseous nerve is a basivertebral nerve.
